(19) United States
(12) Patent Application Publication
(10) Pub. No.: US 2022/0118036 A1
(43) Pub. Date: Apr. 21, 2022

(54) USE OF MORCHELLA ACTIVE SUBSTANCE

(71) Applicant: GRAPE KING BIO LTD., Taoyuan City (TW)

(72) Inventors: Chin-Chu CHEN, Taoyuan City (TW); I-Chen LI, Taoyuan City (TW); Tsung-Ju LI, Taoyuan City (TW); Ting-Yu LU, Taoyuan City (TW); Lynn-Huey CHIANG, Taoyuan City (TW); Yen-Po CHEN, Taoyuan City (TW)

(73) Assignee: GRAPE KING BIO LTD., Taoyuan City (TW)

(21) Appl. No.: 17/198,928

(22) Filed: Mar. 11, 2021

(30) Foreign Application Priority Data
Oct. 16, 2020 (TW) 109135867

(51) Int. Cl. A61K 36/062; A61K 9/19; A61P 21/06

(52) U.S. Cl. CPC A61K 36/062; A61K 9/19; A61P 21/06; A61K 2236/11; A61K 2236/51; A61K 2236/331; A61K 2236/333; A61K 2236/19

(57) ABSTRACT

The present invention provides a novel use of Morchella active substance. The Morchella active substance is for manufacturing a composition for ameliorating Sarcopenia. The preparation method of the Morchella active substance comprises the following steps: (a) culturing Morchella mycelium in a plate media between 15° C. and 30° C. for 1 to 2 weeks; (b) inoculating the Morchella mycelium cultured in step (a) to a flask and culturing the Morchella mycelium between 15° C. and 30° C. with a pH of 2 to 6 for 4 to 7 days; and (c) inoculating the Morchella mycelium cultured in step (b) to a fermentation tank and culturing the Morchella mycelium between 15° C. and 30° C. with a pH of 2 to 6 for 6 to 10 days, so as to obtain a Morchella mycelium fermentation broth containing the Morchella active substance.

### BACKGROUND OF THE INVENTION

### Field of the Invention

The present invention relates to a preparation method and a use of a Morchella active substance; more specifically, to a use of the Morchella active substance to slow down muscle atrophy, increase muscle mass (weight), muscle grip strength and muscle endurance, or prepare a pharmaceutical composition for ameliorating sarcopenia.

### Description of the Prior Art

Skeletal sarcopenia, or sarcopenia for short, refers to a phenomenon in which skeletal muscle mass and strength are lost and physical activity is reduced with age. Generally speaking, after the age of 30, the muscle mass will lose 0.5%-1% every year, about 40% after the age of 75, and 50% at the age of 90. Studies in the United States and some European regions showed that the prevalence rate of sarcopenia is about 5-13% for people aged 60-70, and about 11-50% for people over 80. Research in Taiwan showed that the prevalence rate of sarcopenia in the elderly over 65 is about 3.9-7.3%.

In addition to producing force and motion, muscles have also been identified as an endocrine organ that produces and releases cytokines. Therefore, suffering from sarcopenia may increase the incidence of disease, reduce the quality of life, and even death. However, there is no effective treatment for sarcopenia currently.

Morchella is a world-famous large-scale edible fungus and its fruit body has irregular pits and ridges, like the sheep tripe in shape, so as to be named. Morchella is delicious and nutritious, and its nutrient content is equivalent to milk and fish. Not only is it rich in protein and carbohydrates, it also contains a variety of trace elements and 19 kinds of amino acids. This fungus has the effects of benefiting the intestines and stomach, helping digestion, dispelling phlegm and regulating Qi, and can be used to treat spleen and stomach, indigestion, and shortness of breath.

At present, there is no related research showing that Morchella can be used to ameliorate sarcopenia or increase muscle mass, grip strength and muscle endurance.

### SUMMARY OF THE INVENTION

The present disclosure provides a preparation method of a Morchella active substance, which extracts from a Morchella mycelium in a specific manner and applies its use for a new application.

According to an embodiment of the present disclosure, it provided a method for ameliorating sarcopenia, comprising administrating an effective amount of an active substance of Morchella to a subject. The preparation method of the Morchella active substance comprises the following steps:

(a) culturing a Morchella mycelium in a plate medium between 15° C. and 30° C. for 1 to 2 weeks;

(b) inoculating the Morchella mycelium cultured in step (a) to a flask and culturing the Morchella mycelium between 15° C. and 30° C. with a pH of 2 to 6 for 4 to 7 days; and

(c) inoculating the Morchella mycelium cultured in step (b) to a fermentation tank and culturing the Morchella mycelium with stirring between 15° C. and 30° C. with a pH of 2 to 6 for 6 to 10 days to form a Morchella mycelium fermentation broth containing the Morchella active substance.

According to another embodiment of the present disclosure, it provided a method for increasing muscle mass, grip strength and/or muscle endurance. The method comprises administrating an effective amount of an active substance of Morchella to a subject. The preparation method of the Morchella active substance comprises the following steps:

(a) culturing a Morchella mycelium in a plate medium between 15° C. and 30° C. for 1 to 2 weeks;

(b) inoculating the Morchella mycelium cultured in step (a) to a flask and culturing the Morchella mycelium between 15° C. and 30° C. with a pH of 2 to 6 for 4 to 7 days; and

(c) inoculating the Morchella mycelium cultured in step (b) to a fermentation tank and culturing the Morchella mycelium with stirring between 15° C. and 30° C. with a pH of 2 to 6 for 6 to 10 days to form a Morchella mycelium fermentation broth containing the Morchella active substance.

In one embodiment, the Morchella mycelium is selected from Morchella esculenta, Morchella crassipes, Morchella angusticeps, Morchella conica, Morchella elate, Morchella deliciosa, and a combination thereof.

In one embodiment, the Morchella mycelium is morchella esculenta mycelium deposited at the Food Industry Research and Development Institute, with a deposit number BCRC-36352.

In one embodiment, the preparation method of the Morchella mycelium active substance further comprises a step (d): freeze-drying and then grounding the Morchella mycelium fermentation broth of step (c) to form a Morchella mycelium lyophilized powder containing the Morchella active substance.

In one embodiment, the preparation method of the Morchella mycelium active substance further comprises a step (e): extracting the Morchella mycelium lyophilized powder with at least one solvent to form a Morchella mycelium extract containing the Morchella active substance.

In one embodiment, the solvent is pure water and/or ethanol.

In one embodiment, the preparation method of the Morchella mycelium active substance further comprises a step (f): drying the Morchella mycelium extract to obtain the Morchella active substance.

In one embodiment, sarcopenia has symptoms including muscle atrophy, decreased muscle mass, decreased grip strength and/or decreased muscle endurance.

To make the above and other aspects of the present invention clearer and easier to understand, the following embodiments are given to illustrate in detail with the accompanying drawings.

### BRIEF DESCRIPTION OF THE DRAWINGS

FIG. 1 shows that the water extract of Morchella mycelium can restore the glucocorticoid-dexamethasone induced muscle atrophy of mouse skeletal muscle cells C2C12.

FIG. 2 shows that the ethanol extract of Morchella mycelium can restore the glucocorticoid-dexamethasone induced muscle atrophy of mouse skeletal muscle cells C2C12.

### DETAILED DESCRIPTION OF THE EMBODIMENTS

### Example 1 Morchella Mycelium Cultivation

The Morchella used in this example comes from the Biological Resources Research Center (BCRC) of the Food Industry Research and Development Institute in Taiwan, and the Morchella strain is Morchella esculenta with a deposit number of BCRC-36352. However, the Morchella used in the present invention is not limited to Morchella esculenta, and can also be selected from Morchella crassipes, Morchella angusticeps, Morchella conica, Morchella elate, Morchella deliciosa and a combination of the above species. In other embodiments, the present invention can also use other kinds of Morchella and is not limited thereto.

(1) Plate culture: Morchella mycelia were inoculated on a plate, and cultured at 15-30° C. for 1-2 weeks (in this example, cultured at 25° C. for 7 days). The composition of the plate medium may include Potato Dextrose Agar (PDA), carbon source and nitrogen source, and there was no particular limitation.

(2) Flask culture: After the plate culture was completed, the mycelia were scraped off and inoculate in a flask, and then cultured with shaking between 15° C. and 30° C. with a pH of 2 to 6 and a rotating speed of 110-150 rpm for 4-7 days (In this example, the culture was shaken at 25° C., pH 5, and rotating speed 120 rpm for 7 days). This shaking culture was cultured in the media shown in Table 1 below.

TABLE 1
Medium formulations

| | Content used in | Preferred |
|---|---|---|
| | this embodiment | content |
| Components | (wt %) | range (wt %) |
| Comprehensive | 2 | 1-3 |
| carbon and | | |
| nitrogen source | | |
| Saccharides | 2 | 1-4 |
| Yeast extract | 0.3 | 0.1-1 |
| Peptone | 0.3 | 0.1-1 |
| Inorganic salts | 0.1 | 0.01-0.5 |

In the above medium formulations, the comprehensive carbon and nitrogen sources can be selected from cereals (such as wheat flour) or beans (such as soy powder, mung bean powder, soybean powder, cinnamon powder, etc.); saccharides can be glucose, fructose, maltose, sucrose, etc.; inorganic salts can be magnesium sulfate, dipotassium hydrogen phosphate, potassium dihydrogen phosphate, iron sulfate, etc. In particular, the medium formulation in Table 1 is only one example, and the components can be adjusted according to requirements during use, or used with commercially available media, as there is no special restriction.

(3) Culture in a fermentation tank: the mycelium cultured in the flask in (2) was inoculated into the fermentation tank between 15° C. and 30° C. with a tank pressure of 0.5-1.0 kg/cm², a pH of 2 to 6, a stirring speed of 50 to 150 rpm and an aeration rate of 0.1-1.5 WM for 6-10 days to form a Morchella mycelium fermentation broth (in this example, the condition was set at 25° C., a tank pressure of 0.5 kg/cm², a pH of 5, a stirring speed of 80 rpm and an aeration rate of 1.0 WM (air) for 14 days). The culture medium used in the fermentation tank could be the same as the culture medium used in the flask culture in step (2). This Morchella mycelium fermentation broth contains the Morchella mycelium active substance of the present invention. The Morchella mycelium fermentation broth can be further prepared as the lyophilized powder of the Morchella mycelium fermentation broth by a freeze-drying step. In this example, about 3 kg of lyophilized powder can be obtained from 100 L of Morchella mycelium fermentation broth.

(4) Preparation of extract: pure water or ethanol was used as the solvent to extract the lyophilized powder of (3).

Water extract: First, 200 g lyophilized powder of Morchella mycelium fermentation broth was added into distilled water, of which the volume was 20 times the volume of the lyophilized powder, and then heated at 100° C. for 30 minutes to dissolve the lyophilized powder. After cooling, the freeze-drying process was carried out to obtain the water extract of Morchella mycelium fermentation broth.

Ethanol extract: 200 g lyophilized powder of Morchella mycelium fermentation broth was added into ethanol, of which the weight was 20 times the weight of the lyophilized powder, shaken with ultrasound for 1 hour, and then filtered to obtain the supernatant. The supernatant was concentrated under reduced pressure to obtain the ethanol extract of Morchella mycelium fermentation broth.

Through the extraction step, a higher concentration of Morchella active substance can be obtained. The types of the Morchella active substance include Morchella mycelium fermentation broth (mycelium and clear liquid/supernatant), fermentation broth lyophilized powder, water extract, ethanol extract, a mixture of water extract and ethanol extract, lyophilized powder of water extracts or ethanol extracts or other dosage forms. In Example 2 below, water and ethanol extracts are used as the types of the Morchella active substance to ameliorate muscle atrophy. In Example 3, the water extract and the ethanol extract are thoroughly mixed to obtain the “extract mixture”, which is used as the Morchella active substance in the feeding test. In one embodiment, the mixing ratio of the water extract and the ethanol extract is 1:1 by weight.

### Example 2 Amelioration of Muscle Atrophy by Morchella Active Substance (Water Extract and Ethanol Extract)

Currently, there have been experiments using Dexamethasone to induce muscle atrophy in mouse skeletal muscle cell C2C12. Please refer to the paper conducted by Chen-Yuan Chiu, Rong-Sen Yang, Meei-Ling Sheu, Ding-Cheng Chan, Ting-Hua Yang, Keh-Sung Tsai, Chih-Kang Chiang, Shing-Hwa Liu, “Advanced glycation end-products induce skeletal muscle atrophy and dysfunction in diabetic mice via a RAGE-mediated, AMPK-down-regulated, AKt pathway”, J Pathol, 2016 February; 238(3):470-82. This paper uses a synthetic glucocorticoid-Dexamethasone to induce differentiated C2C12 atrophy as a model of muscle atrophy for sarcopenia study, in which the severity of muscle atrophy is measured according to the observation of morphology and diameter of the myotubes. According to such a method, in this experiment, the differentiated mouse skeletal muscle cell C2C12 is stained with H&E (hematoxylin and eosin) to assess the effect of the Morchella mycelium active substance in muscle atrophy. The experimental method is as follows:

(1) After the mouse skeletal muscle cells C2C12 was collected and the cell suspension concentration was adjusted, the cells were plated in a 6-well plate at a cell density of 1×10⁵-2×10⁵ with the cell growth medium (DMEM, 10% Fetal Bovine Serum).

(2) The 6-well plate of (1) was placed in a 5% CO₂ incubator at 37° C. for two days and then observed under a microscope. When the cells grew to 70% confluency, a differentiation medium (DMEM, 2% Horse Serum) was added for induction. The fresh differentiation medium was replaced every two days. The differentiation process was seven days in total.

(3) On the seventh day of cell differentiation, after about 90% of the cells formed myotubes, 1 μM Dexamethasone and a single dose of Morchella mycelium water extract (10 μg/ml) or ethanol extract (1 μg/ml) obtained in Example 1 were added. The extract of Morchella mycelium was dissolved in Dimethyl sulfoxide (DMSO), and the concentration of DMSO added was adjusted as 0.1% during the test to prevent its toxicity from affecting cell growth. The control group was treated with 0.1% DMSO and all experimental and control groups were performed in triplicates. After incubating cells in a 5% CO₂ incubator at 37° C. for 24 hours, subsequent testing experiments were performed.

(4) When the myotube was atrophied, its width would be significantly reduced. Using an inverted microscope under a 40× magnified field of view, 60 cells were taken for each repetition, and Image-Pro Plus software was used to calculate the width of the myotube to determine the degree of muscle atrophy.

(5) Each test was repeated 3 times, and the numerical value was expressed as mean±SD (standard deviation). The statistical method used paired t-test to analyze the percentages of each item, and the significant difference was determined when the p-value was less than 0.05.

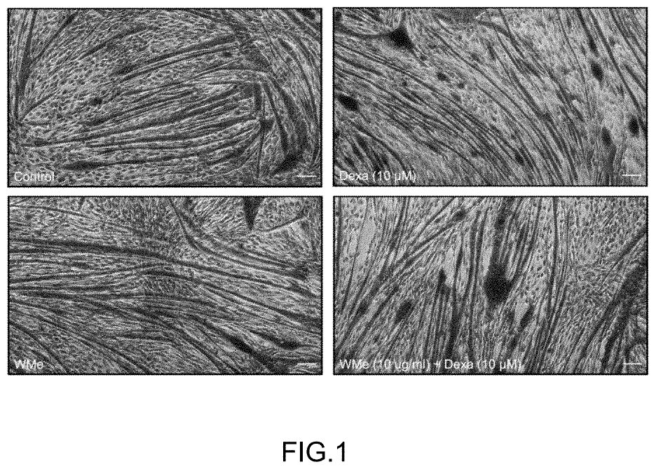

(6) The experimental results of Morchella mycelium water extract (WMe) are shown in FIG. 1 and Table 1 below. In the picture, the dark red part is the myotube. The C2C12 myotube treated with Dexamethasone (upper right) has a significantly smaller diameter (30.39 μm→19.98 μm) than that of the control group (upper left), which proves that this model does cause muscle atrophy. The addition of Morchella mycelium water extract can significantly restore Dexamethasone-induced reduced muscle diameter (bottom right, 19.98 μm→29.37 μm). Moreover, treatment with Morchella mycelium water extract alone (bottom left) has no significant effect on muscle mass.

TABLE 1
Test results of Morchella mycelium water extract

| Groups | myotube diameter (μm) |
|---|---|
| Control | 30.39 ± 4.52 |
| Dexamethasone (Dex) | 19.98 ± 6.42# |
| Dex + WMe | 29.37 ± 7.36* |
| WMe | 30.90 ± 5.92* |

(n = 60)
#Indicating a statistically significant difference from the control group (p < 0.05)
*Indicating a statistically significant difference from the Dex group (p < 0.05)

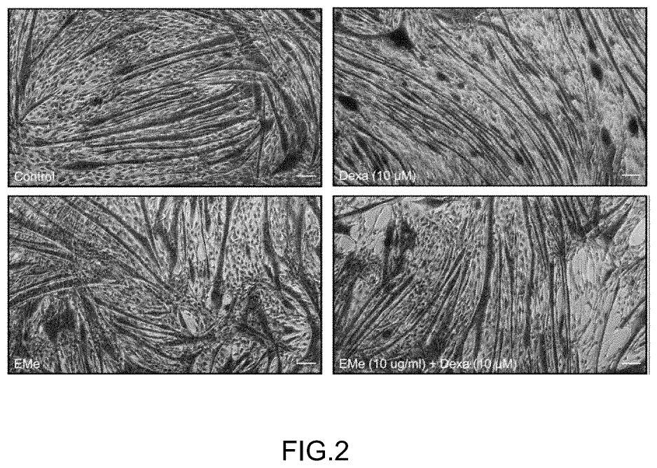

(7) The experimental results of Morchella mycelium ethanol extract (ethanol, EMe) are shown in FIG. 2 and Table 2 below. The C2C12 myotube treated with Dexamethasone (upper right) has a significantly smaller diameter than that of the control group (upper left). The addition of Morchella mycelium ethanol extract can significantly restore muscle diameter reduced by dexamethasone (19.98→30.70 μm, bottom right). Moreover, treatment with Morchella mycelium ethanol extract alone has no significant effect on muscle mass (bottom left).

TABLE 2
Test results of Morchella mycelium ethanol extract

| Groups | myotube diameter (μm) |
|---|---|
| Control | 30.39 ± 4.52 |
| Dexamethasone (Dex) | 19.98 ± 6.42# |
| Dex + EMe | 30.70 ± 6.24* |
| EMe | 28.80 ± 6.34* |

(n = 60)
#Indicating a statistically significant difference from the control group (p < 0.05)
*Indicating a statistically significant difference from the Dex group (p < 0.05)

The above experiments confirmed that the Morchella active substance of the present invention has the effect of ameliorating Dexamethasone-induced muscle atrophy, which can be combined with the pharmaceutically acceptable carrier, excipient, diluent or adjuvant to prepare the pharmaceutical composition, or can also be used as the food additive for the prevention/treatment of sarcopenia.

### Example 3 Increase of Muscle Mass, Muscle Grip Strength and Muscle Endurance by Morchella Active Substance (Water/Ethanol Extract Mixture)

At present, there have been experiments using cast immobilization (IM, using a fixed mechanical mechanism to fix the hind limbs of mice to cause muscle atrophy) to induce mouse skeletal muscle atrophy. The paper of Peter Bialek, Carl Morris, Jascha Parkington, Michael St. Andre, Jane Owens, Paul Yaworsky, Howard Seeherman, and Scott A. Jelinsky, “Distinct protein degradation profiles are induced by different disuse models of skeletal muscle atrophy”, Physiological Genomics, 2011 October; 43(19):1075-1086 can be referred. In this experiment, the same method was used to induce skeletal muscle atrophy in mice by cast immobilization (IM). After IM was induced for 7 days, the IM was removed, the muscle grip strength test was measured, and then the muscle endurance test was measured after the mice were forced to run on the treadmill at a fixed rate of 18-20 m/min up to 30 min to avoid electric shock (according to the times of electric shock). At the end of the test, the mice were sacrificed, and the skeletal muscles of the hind limbs were removed, weighed and recorded to evaluate the results of the Morchella mycelium extract mixture on increasing muscle mass, muscle grip strength and muscle endurance.

(1) C57BL/6J mice were used for animal experiments, and divided into a control group (Sham group), an IM group and a Morchella mycelium extract mixture group, with 6 mice in each group. The mice in the control group and the IM group were fed equal volumes of secondary pure water (ddH₂O), and the test substance (i.e., the water/ethanol extract mixture of Morchella mycelium) was fed at a concentration of 500 mg/kg. The test substance was fed orally by a gastric tube, and the used volume was 10 mL/kg b.w./time. The test substance was freshly prepared before administration. The mice were observed continuously after daily feeding, and their body weight, food intake and water intake were recorded.

(2) The mice were treated with cast immobilization (IM) to induce sarcopenia and simultaneously given the water/ethanol extract mixture of Morchella mycelium for 14 days. Afterward, the mice were tested for muscle grip strength and muscle endurance (according to the times of electric shock). At the end of the test, the mice were sacrificed, and the skeletal muscles of the hind limbs were removed, weighed and recorded.

The test results are all expressed in Mean±SD, and GraphPad Prism (version 8.0) is used for statistical analysis of the experimental data. As to the statistical methods, one-way ANOVA and post-hoc Dunnett's test are used to determine differences between groups. If p-value of the statistical result is less than 0.05 (p<0.05), it is determined that there is a significant difference between the two groups.

The results of the mouse muscle grip strength test are shown in Table 3 below. The muscle grip strength test measures the maximum grip strength of mice, and a higher value is better. On the 14th day, the IM group had lower muscle grip strength than the Sham group, and the Morchella mycelium water/ethanol extract mixture group has significantly increased muscle grip strength than the IM group.

TABLE 3
Muscle grip strength test results of
Morchella mycelium extract mixture

| | Muscle grip |
|---|---|
| Groups | strength (Force) |
| Control (Sham) | 154.38 ± 21.28 |
| Cast Immobilization (IM) | 106.67 ± 22.79# |
| IM + Morchella mycelium | 118.43 ± 24.94* |
| water/ethanol extract mixture | |

(n = 6)
#Indicating a statistically significant difference from the control group (Sham) (p < 0.05)
*Indicating a statistically significant difference from the IM group (p < 0.05)

The results of the mouse muscle endurance test are shown in Table 4 below. The more times of electric shock recorded, the worse the endurance will be. On the 14th day, the muscle endurance of the group treated with IM decreases significantly, as the times of electric shock recorded increased (3.00→500.83). However, the times of electric shock recorded in the Morchella mycelium water/ethanol extract mixture is significantly lower than that in the IM group (500.83→78.50).

TABLE 4
Muscle endurance (electric shock times) test
results of Morchella mycelium extract mixture

| | Muscle endurance |
|---|---|
| Groups | (electric shock times) |
| Control (Sham) | 3.00 ± 2.28 |
| Cast Immobilization (IM) | 500.83 ± 257.37# |
| IM + Morchella mycelium | 78.50 ± 135.18* |
| extract mixture | |

(n = 6)
#Indicating a statistically significant difference from the control group (Sham) (p < 0.05)
*Indicating a statistically significant difference from the IM group (p < 0.05)

After the mice were sacrificed, the skeletal muscles of their hind limbs were removed and weighed. The weight measurement results are shown in Table 5 below. The weight of the gastrocnemius muscle of IM is lower than that of the control group (Sham group), while the Morchella mycelium water/ethanol extract mixture group significantly increased gastrocnemius muscle weight when compared with the IM group. These findings proved that the Morchella mycelium water/ethanol extract mixture of the present invention can increase muscle weight.

TABLE 5
Gastrocnemius muscle weight test results
of Morchella mycelium extract mixture

| | Gastrocnemius muscle |
|---|---|
| Groups | weight (mg/g BW) |
| Control (Sham) | 5.485 ± 0.234 |
| Cast Immobilization (IM) | 4.895 ± 0.384# |
| IM + Morchella mycelium | 5.124 ± 0.385* |
| extract mixture | |

(n = 6)
#Indicating a statistically significant difference from the control group (Sham) (p < 0.05)
*Indicating a statistically significant difference from the IM group (p < 0.05)

### Example 4 Preparation of Composition

The above experiments confirmed that the Morchella active substance (water/ethanol extract mixture) of the present invention has the effect of increasing muscle mass, muscle grip strength and muscle endurance, and could be used as a new application of Morchella in the field of medicine. Accordingly, to specifically apply the active substance of Morchella, a composition containing the Morchella active substance is manufactured, and an effective amount thereof is administered to a subject to achieve the healing effect.

The “effective amount” described above refers to an amount that is sufficient to produce the aforementioned effects of improving sarcopenia and/or increasing muscle mass, grip strength and/or muscle endurance. Based on in vitro cell culture experiments, the aforementioned effective amount is defined as “μg/ml” based on the total volume of cell culture medium used in each culture. Based on animal model experiments, the aforementioned effective amount is defined as “g/60 kg body weight/day.” In addition, the data of effective amount obtained via in vitro cell culture experiments can be converted to a reasonable effective amount for animal use by the following formula:

In general (Reagan-Shaw et al., 2008), 1 “μg/ml” units (based on the effective amount of in vitro cell culture experiments) may be equivalent to 1 “mg/kg body weight/day” units (based on the effective amount of rat model experiments), and, based on that metabolic rate of a rat is six times of that of a human, the effective human dose can be found.

Therefore, an effective amount for use in mice based on an in vitro cell culture experiment of 500 μg/ml is calculated as 500 mg/kg body weight/day (i.e., 0.5 g/kg body weight/day). Further, taking into account the differences in the aforementioned metabolic rates, an effective amount for human use may be taken as 5 g/60 kg body weight/day.

Based on the test results reported above, a validated dose based on a rat experiment is 500 mg/kg body weight/day and, therefore, a reasonably effective dose for human use should be 2.4 g/60 kg body weight/day.

In one embodiment, an effective amount of the Morchella active substance contained in the composition is 500 mg/60 kg to 10 g/60 kg body weight/day.

The composition further comprises an additive. In a preferred embodiment, the additive may be an excipient, a preservative, a diluent, a filler, an absorption enhancer, a sweetener, or a combination thereof. The excipient can be selected from sodium citrate, calcium carbonate, calcium phosphate, or a combination thereof. This preservative, such as benzyl ethanol, parabens, extends the shelf life of pharmaceutical compositions. The diluent can be selected from water, ethanol, propylene glycol, glycerol, or a combination thereof. The filler can be selected from lactose, nougat, ethylene glycol of high molecular weight, or a combination thereof. Absorption enhancers may be selected from dimethylsulfoxide (DMSO), laurocapram, propylene glycol, glycerol, polyethylene glycol, or a combination thereof. The sweetener may be selected from Acesulfame K, aspartame, saccharin, sucralose, neotame, or a combination thereof. In addition to the additives listed above, other additives that are suitable for use may be selected based on requirements without affecting the medical effect of the Morchella active substance.

The composition can be developed as a different product in the medical field. In a preferred embodiment, the composition is a pharmaceutical product, feed, beverage, nutritional supplement, dairy product, food, or health food.

The composition may take different forms depending on the needs of the receiver. In a preferred embodiment, the composition is in the form of powder, lozenge, granulation, suppository, microcapsule, ampoule/ampule, liquid spray, or suppository.

The composition of the invention can be used in animals or humans. Without affecting the effect of the Morchella active substance, the composition comprising the Morchella active substance can be made into any pharmaceutical form and administered to the animal or human in a suitable manner depending on the type of the drug.

Composition 1: The water extract (20 wt %) was taken as the Morchella active substance, mixed well with benzyl ethanol (8 wt %) as a preservative, glycerin (7 wt %) as a diluent, and dissolved in purified water (65 wt %) to produce a pharmaceutical composition of the present invention in liquid form. The aforementioned wt % means the ratio of each ingredient to the total weight of the composition. Store at 4° C. for later use.

Composition 2: The ethanol extract (15 wt %) was used as the Morchella active substance, mixed well with benzyl ethanol (5 wt %) as a preservative, glycerin (10 wt %) as a diluent, and dissolved in purified water (70 wt %) to produce a pharmaceutical composition of the present invention in liquid form. The aforementioned wt % means the ratio of each ingredient to the total weight of the composition. Store at 4° C. for later use.

Although the present invention has been disclosed above with embodiments, it is not intended to limit the present invention. People having ordinary skill in the art, after considering the above teachings, can make appropriate modifications to the content of the above embodiments, and still achieve the effects claimed in this application. Therefore, the scope of the present invention shall be subject to the claims attached thereafter.

What is claimed is:

1. A method for ameliorating sarcopenia, comprising administrating an effective amount of an active substance of Morchella to a subject, wherein a preparation method of the Morchella active substance comprises the following steps:
(a) culturing a Morchella mycelium in a plate medium between 15° C. and 30° C. for 1 to 2 weeks;
(b) inoculating the Morchella mycelium cultured in step (a) to a flask and culturing the Morchella mycelium between 15° C. and 30° C. with a pH of 2 to 6 for 4 to 7 days; and
(c) inoculating the Morchella mycelium cultured in step (b) to a fermentation tank and culturing the Morchella mycelium with stirring between 15° C. and 30° C. with a pH of 2 to 6 for 6 to 10 days to form a Morchella mycelium fermentation broth containing the Morchella active substance.

2. The method of claim 1, wherein the Morchella mycelium is selected from Morchella esculenta, Morchella crassipes, Morchella angusticeps, Morchella conica, Morchella elate, Morchella deliciosa, and a combination thereof.

3. The method of claim 2, wherein the Morchella mycelium is morchella esculenta mycelium deposited at the Food Industry Research and Development Institute, with a deposit number BCRC-36352.

4. The method of claim 2, wherein the preparation method of the Morchella mycelium active substance further comprises a step (d): freeze-drying and then grounding the Morchella mycelium fermentation broth of step (c) to form a Morchella mycelium lyophilized powder containing the Morchella active substances.

5. The method of claim 4, wherein the preparation method of the Morchella mycelium active substance further comprises a step (e): extracting the Morchella mycelium lyophilized powder with at least one solvent to form a Morchella mycelium extract containing the Morchella active substances.

6. The method of claim 5, wherein the solvent is pure water and/or ethanol.

7. The method of claim 6, wherein the preparation method of the Morchella mycelium active substance further comprises a step (f): drying the Morchella mycelium extract to obtain the Morchella active substances.

8. The method of claim 1, wherein the sarcopenia has symptoms including muscle atrophy, decreased muscle mass, decreased grip strength and/or decreased muscle endurance.

9. A method for increasing muscle mass, grip strength and/or muscle endurance, comprising administrating an effective amount of an active substance of Morchella to a subject, wherein a preparation method of the Morchella active substance comprises the following steps:
(a) culturing a Morchella mycelium in a plate medium between 15° C. and 30° C. for 1 to 2 weeks;
(b) inoculating the Morchella mycelium cultured in step (a) to a flask and culturing the Morchella mycelium between 15° C. and 30° C. with a pH of 2 to 6 for 4 to 7 days; and
(c) inoculating the Morchella mycelium cultured in step (b) to a fermentation tank and culturing the Morchella mycelium with stirring between 15° C. and 30° C. with a pH of 2 to 6 for 6 to 10 days to form a Morchella mycelium fermentation broth containing the Morchella active substance.

10. The method of claim 9, wherein the Morchella mycelium is selected from Morchella esculenta, Morchella crassipes, Morchella angusticeps, Morchella conica, Morchella elate, Morchella deliciosa, and a combination thereof.

11. The method of claim 10, wherein the Morchella mycelium is morchella esculenta mycelium deposited at the Food Industry Research and Development Institute, with a deposit number BCRC-36352.

12. The method of claim 10, wherein the preparation method of the Morchella mycelium active substance further comprises a step (d): freeze-drying and then grounding the Morchella mycelium fermentation broth of step (c) to form a Morchella mycelium lyophilized powder containing the Morchella active substances.

13. The method of claim 12, wherein the preparation method of the Morchella mycelium active substance further comprises a step (e): extracting the Morchella mycelium lyophilized powder with at least one solvent to form a Morchella mycelium extract containing the Morchella active substances.

14. The method of claim 13, wherein the solvent is pure water and/or ethanol.

15. The method of claim 13, wherein the preparation method of the Morchella mycelium active substance further comprises a step (f): drying the Morchella mycelium extract to obtain the Morchella active substances.
